(19) United States
(12) Patent Application Publication
(10) Pub. No.: US 2026/0216495 A1
(43) Pub. Date: Jul. 30, 2026

(54) ELASTIC MECHANISM FOR PREVENTING ACCIDENTAL INTRAVASCULAR CATHETER DISLODGEMENT

(71) Applicant: ABIOMED, Inc., Danvers, MA (US)

(72) Inventor: Christopher Sheils, Danvers, MA (US)

(73) Assignee: ABIOMED, Inc., Danvers, MA (US)

(21) Appl. No.: 19/460,935

(22) Filed: Jan. 27, 2026

Related U.S. Application Data
(60) Provisional application No. 63/750,409, filed on Jan. 28, 2025.

(51) Int. Cl. A61M 60/861; A61M 60/13; A61M 60/865; A61M 60/90

(52) U.S. Cl. CPC A61M 60/861; A61M 60/13; A61M 60/865; A61M 60/90; A61M 2209/088; A61M 2210/125

(57) ABSTRACT

Provided herein is an accessory for use with a catheter of a blood pump system and a method of use. The accessory may include a first end attached to a repositioning unit of a blood pump and a second end. The accessory is configured to move when a force is applied while the catheter of the blood pump system does not move. The method may comprise applying a force to an accessory to a blood pump system and then moving the accessory in a first direction while the blood pump system does not move in the first direction.

### CROSS-REFERENCE TO RELATED APPLICATIONS

The present application claims priority to U.S. Provisional Patent Application No. 63/750,409, filed Jan. 28, 2025, the contents of which are incorporated by reference here in its entirety.

### TECHNICAL FIELD

Embodiments disclosed herein relate to medical devices, such as catheter-based intravascular blood pumps, devices and methods.

### BACKGROUND

Currently, percutaneous mechanical support devices are leveraged for a variety of clinical indications. Such support devices may comprise, but are not limited to, an Impella® pump, an Extracorporeal Membrane Oxygenation (ECMO) pump, and a balloon pump. The Impella® pump may further comprise an Impella CP® pump, Impella 5.5® pump, Impella RP® pump, and/or an Impella RP Flex® pump, all of which are by Abiomed, Inc. of Danvers, Mass. Most often they are inserted into a patient percutaneously through a single access point (e.g., radial access, femoral access, axillary access) while other procedures, such as, for example, percutaneous coronary intervention (PCI) are performed through a second access point, such as a contralateral femoral or radial access point. The use of multiple devices on a patient at the same time therefore often requires multiple access sites which presents several challenges.

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the “Impella” heart pump. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### SUMMARY OF INVENTION

In some embodiments, an accessory for use with a blood pump system may be provided. The blood pump system may have a blood pump, a catheter attached to the blood pump, and a repositioning unit configured to receive at least a portion of the blood pump and/or catheter and to secure the blood pump system to the patient. The accessory may comprise a first end configured to be attached to a portion of the repositioning unit. The accessory may also comprise a second end. When the accessory is grasped and when a force is applied to the accessory, the accessory may be moveable in a first direction of the force while the blood pump system does not move in a first direction.

In some embodiments, an accessory for use with a blood pump system having a blood pump, a catheter attached to the blood pump, a repositioning unit configured to receive at least a portion of the blood pump and/or catheter and to secure the catheter to the patient may be provided. The accessory may comprise a body configured to be slidable over the catheter. The body may have a first end configured to be attached to a portion of the blood pump system and a second end. The second end of the body may be moveable relative to the first end upon application of a force.

In some embodiments, a method may be provided. The method may include applying a force to an accessory to a blood pump system. The accessory may have a body configured to be slidable over a catheter. The body may have a first end configured to be attached to a portion of the blood pump system and a second end. The second end of the body may be moveable relative to the first end. The method may include moving the accessory in a first direction while the blood pump system does not move in the first direction.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 shows an illustrative view of a blood pump inserted into the left ventricle;

FIG. 2 shows an illustrative view of a blood pump inserted into the right ventricle;

FIG. 3 shows an illustrative side view of a portion of a repositioning unit;

FIG. 4 shows an illustrative side view of an accessory;

FIG. 5A shows an illustrative side view of an accessory;

FIG. 5B shows an illustrative side view of an accessory;

FIG. 5C shows an illustrative side view of an accessory;

FIG. 5D shows an illustrative side view of an accessory;

FIG. 6 shows an illustrative side view of an accessory;

FIG. 7A shows an illustrative side view of an accessory;

FIG. 7B shows an illustrative side view of an accessory; and

FIG. 8 shows a method of preventing catheter dislocation.

### DETAILED DESCRIPTION

As it is known, percutaneous blood pumps may be inserted into a patient's vasculature to assist with cardiovascular function. In such instances, the blood pumps may be configured to move blood from a first location to a second location. In one exemplary embodiment where the blood pump is placed in the left ventricle, the pump may be configured to move oxygenated blood from the left ventricle into the aorta. As it is known, such left-sided pumps may be inserted via the femoral artery, axillary artery, or direct mediastinal access and positioned across the patient's aortic valve. In another exemplary embodiment where the blood pump is placed in the patient's right ventricle, the pump may be configured to move blood from the inferior vena cava into the pulmonary valve. As it is known, such right-sided pumps may be inserted via the femoral vein or internal jugular vein and positioned across the patient's pulmonary valve.

As it is also known, such blood pumps and catheters need to be in their proper position to function appropriately and benefit the patient. During support, it is possible the blood pump and/or catheter may move within the patient. It is also possible the blood pump and/or catheter may move due to the patient ambulating and/or moving during support. As it is known, some devices may apply a load to the catheter to maintain position. However, in some instances, unintended dislodgements of such devices may occur due to movement of the catheter at the access point. Accordingly, the inventors have recognized the benefits of providing a blood pump with unintended dislodgement mechanisms at the access site to mitigate and/or prevent movement of the blood pump during support.

According to one aspect of the present disclosure, an accessory for use with a blood pump system may be provided. The blood pump system may have a blood pump, a catheter attached to the blood pump, and a repositioning unit. The repositioning unit may be configured to receive at least a portion of the blood pump and/or the catheter. The repositioning unit also may secure the blood pump system to the patient. The accessory may have a first end and a second end. The first end may be configured to be attached to a portion of the repositioning unit. When the accessory is grasped by the patient and when a force is applied, the accessory may be moveable in a first direction of the force while the blood pump system does not move in the first direction of the force.

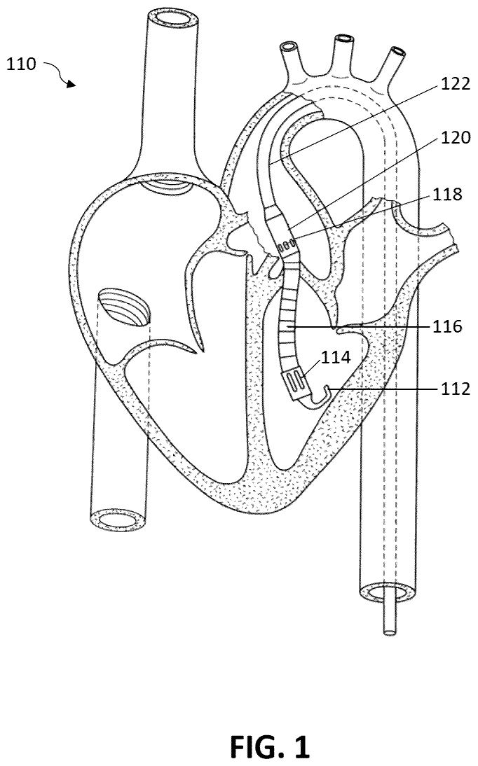

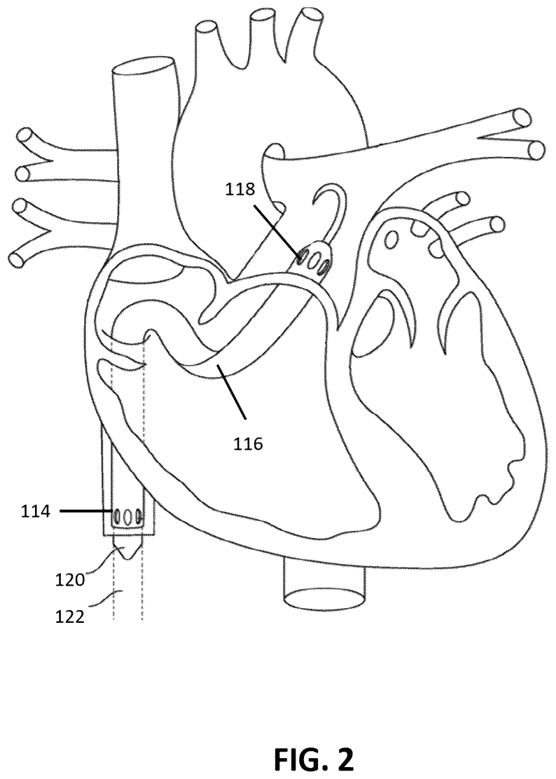

Turning to the figures, FIG. 1 shows a view of a blood pump 110 of a blood pump system. The blood pump 110 may include an atraumatic tip 112, a blood flow inlet 114, a cannula 116, a blood flow outlet 118, a blood pump 120, and a catheter 122. In some embodiments, the blood pump 100 may be positioned in the left ventricle of a patient, as shown in FIG. 1. In other embodiments, the blood pump 100 may be positioned in the right ventricle of a patient, as shown in FIG. 2. In the embodiment shown in FIG. 2, the blood flow outlet 118 may be positioned in the pulmonary artery of a patient and the blood flow inlet 114 may be positioned in the inferior or superior vena cava of a patient. In some embodiments, the cannula 116 of the blood pump shown in FIG. 2 may have one or more preformed bends to accommodate patient anatomy. In other embodiments, the cannula 116 of the blood pump shown in FIG. 2 may be flexible and thus able to bend inside of the patient to accommodate different patient anatomy.

The blood pump system of FIG. 1 may be inserted percutaneously or surgically. For example, when used for left heart support, the blood pump system may be inserted via a catheterization procedure through the femoral artery or axillary artery, into the aorta, across the aortic valve, and into the left ventricle. Once positioned in this way, the blood pump system may deliver blood from the blood inlet 114, which sits inside the left ventricle, through cannula 116, to the blood flow outlet 118, which sits inside the ascending aorta.

Like the blood pump system shown in the embodiment in FIG. 1, the blood pump system of FIG. 2 may also be inserted percutaneously. For example, when used for right heart support, the blood pump system of FIG. 2 may be inserted via a catheterization procedure through the femoral vein, into the inferior vena cava, through the right atrium, across the tricuspid valve, into the right ventricle, through the pulmonary valve, and into the pulmonary artery. Once positioned in this way, the blood pump system of FIG. 2 may deliver blood from the blood inlet 114, which sits inside the inferior vena cava, through the cannula 116, to the blood flow outlet 118, which sits inside the pulmonary artery. In some embodiments, the blood pump system of FIG. 2 may be inserted via the internal jugular (IJ) vein. For example, in such embodiments, intracardiac blood pump system may be inserted via a catheterization procedure through the IJ vein, into the superior vena cava, through the right atrium, across the pulmonary valve, and into the pulmonary artery. Once positioned in this way, the intracardiac blood pump system may deliver blood from the blood flow inlet 114, which sits inside the superior vena cava, through cannula 116, to the blood flow outlet 118, which sits inside the pulmonary artery.

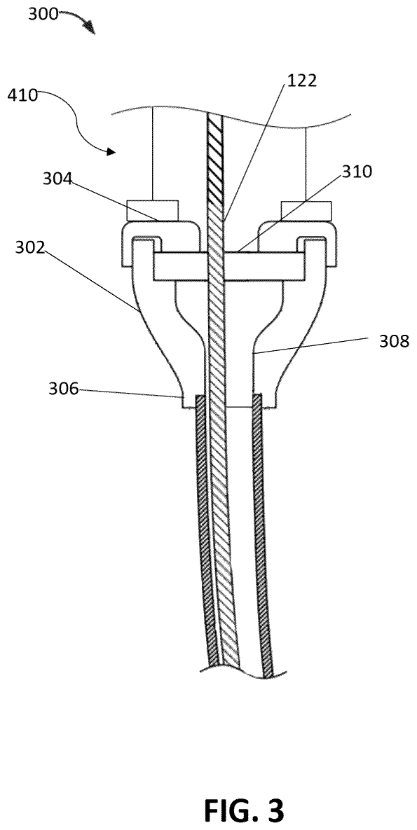

Turning to FIG. 3, a portion of the catheter 122 can be seen passing through a repositioning unit 300. As shown in this view, the repositioning unit 300 may be a sheath comprising a tubular body 302. The tubular body may comprise a proximal body 304 and a distal body 306. The tubular body may define a lumen 308. At least a portion of the distal body 306 may be inserted into a patient's vasculature. The catheter 122 may be inserted through the lumen 308. At least a portion of the catheter 122 may enter the repositioning unit 300. The repositioning unit 300 may be secured to the patient via suturing or any other suitable means. In some embodiments, the repositioning unit 300 may comprise a hemostatic valve 310.

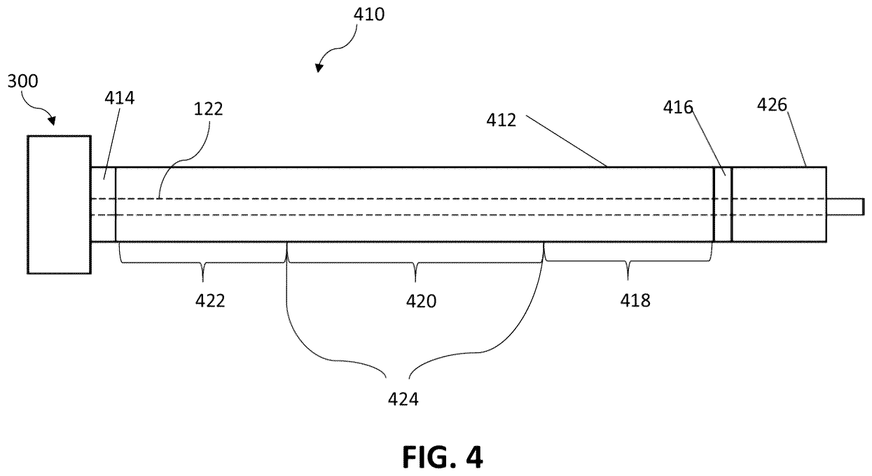

As illustrated in FIG. 3, an accessory 410 of an accessory system may surround at least a portion of the catheter 122 that is proximal to the repositioning unit 300. The accessory system is illustrated in FIG. 4. The accessory system may comprise a body 412. The body 412 may extend along at least a portion of the longitudinal axis of the catheter 122. The body 412 may have a first end 414 and a second end 416 on opposing ends. The first end 414 may be attached to a portion of the repositioning unit 300. In some embodiments, the second end 416 may be freely movable. In other embodiments, the second end 416 may be attached to an anti-contamination sleeve 426. The anti-contamination sleeve 426 surrounds the catheter 122. The anti-contamination sleeve 426 extends along at least a portion of the length of the catheter 122. In some embodiments, the anti-contamination sleeve 426 may extend along the entire length of the catheter 122 from the second end 416 to the patient console (not shown). When the accessory is grasped by a patient and thus a force is applied to the accessory system 400, the body 412 may be moveable in a direction of the force while the catheter 122 of the blood pump does not move in the direction of the force. In some embodiments, the body 412 and the second end 416 may be moveable in a direction of the force while the catheter 122 of the blood pump does not move. In other embodiments, the body 412 may extend along the entire length of the catheter 122 and connect to the patient console (not shown).

The body 412 may further comprise a proximal portion 418, an intermediate portion 420, and a distal portion 422. The intermediate portion 420 may extend distally from the proximal portion 418. The distal portion 422 may extend distally from the intermediate portion 420. In some embodiments, portions of the body 412 may be different materials. For example, each of the proximal portion 418, the intermediate portion 420, and the distal portion 422 may be different materials, respectively. In one embodiment, the proximal portion 418 and the distal portion 422 may be made of a first material, while the intermediate portion 420 may be made of a second material. For example, in some embodiments the proximal portion 418 and the distal portion 422 may be made of a viscoelastic material while the intermediate portion 420 may be made of an elastic polymer material. In other embodiments, the proximal portion 418 and the distal portion 422 may be made of an elastic polymer material and the intermediate portion 420 may be made of a viscoelastic material. Each of the portions of the body 412 may be made of any suitable material. The body 412 may also comprise any number of suitable portions.

In one embodiment, the proximal portion 418 may be made of a material that extends at a load less than the load required to extend the material of the intermediate portion 420. In other embodiments, the distal portion 422 may be made of a material that extends at a load less than the load required to relocate the portion of the body 420 in the repositioning unit 300.

A transition region 424 may be positioned at the point between two adjacent portions. For example, as shown in FIG. 4, there may be a transition region 424 between the proximal portion 418 and the intermediate portion 420 as well as between the intermediate portion 420 and the distal portion 422. As will be appreciated, although two transition regions 424 are shown in the embodiment in FIG. 4, any suitable number of transition regions 424 may be used. In some embodiments, the materials of adjacent portions may blend via any suitable means. For example, in one embodiment, the materials may blend via layering with varying thicknesses. Alternatively, in some embodiments, the materials may blend into a homogeneous mixture. In other embodiments, the materials of adjacent portions may have distinct borders and thus may not blend.

In some embodiments, the first end 414 may be removably detached to the repositioning unit 300. The first end 414 may be removably detached to the repositioning unit 300 via any suitable means, including but not limited to clips, J-locks, etc. In some embodiments, the first end 414 may be disconnected from the repositioning unit 300 without removing the catheter 122 from the patient. In other embodiments, the first end 414 may be removably detached from the second end 416 and configured such that when a force is applied to the accessory 410, the second end 416 is detached from the first end 414. In another embodiment, the first end 414 may be integrally formed with the repositioning unit 300. In another embodiment, the second end 416 may be attached to the anti-contamination sleeve 426. The second end 416 may be attached to the anti-contamination sleeve 426 via any suitable means, including but not limited to clips, J-locks, etc.

In some embodiments, the body 412 may be a tubular member, as shown in the exemplary embodiment in FIG. 4. The tubular member may completely surround the entire circumferential space around the catheter 122. The tubular member may be a constant diameter along the length of the body 412. In other embodiments, the tubular member may have a varying diameter along the length of the body 412. While diameter is used, there is no requirement that the body 412 have a circular cross-section. In some embodiments, diameter may refer to the largest cross-sectional measurement. For example, in some embodiments, the body 412 may be an oval shape and thus the diameter may refer to the largest cross-sectional measurement. As will be appreciated, although described as having a circular cross-section, the body 412 may have any suitable cross-sectional geometry. For example, the body may have a quadrilateral cross-sectional geometry.

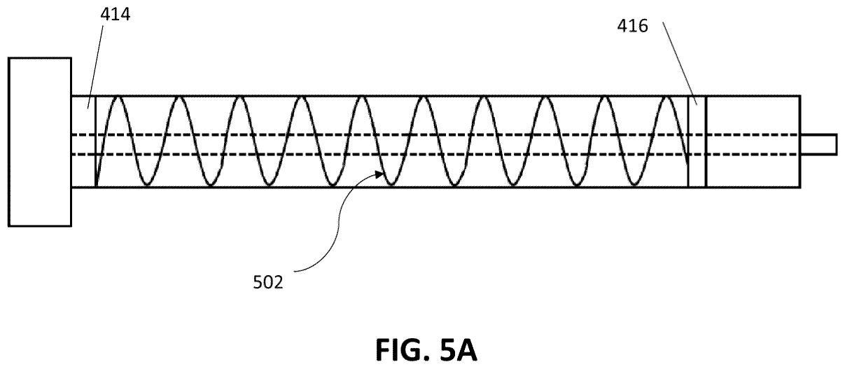

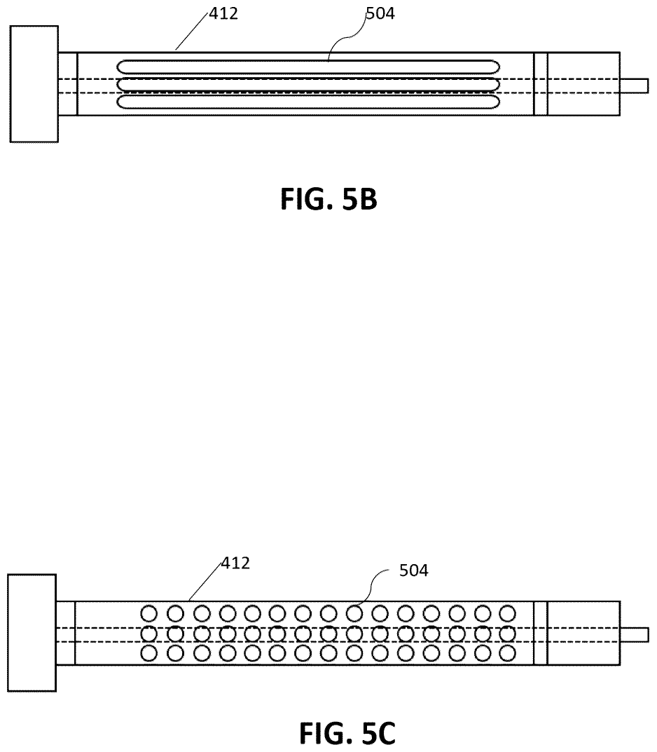

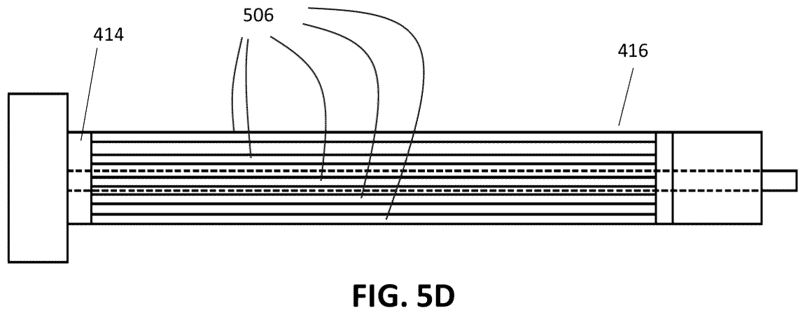

In other embodiments, first end 414 and the second end 416 may be connected by a spring 502, as shown in FIG. 5A. In some embodiments, the spring 502 may be made of a metal material. In other embodiments, the spring 502 may be made of a polymer material. In some embodiments, the spring 502 may be made of a mixture of materials. In some embodiments, the spring 502 may be configured to rest with the coils close together. In such embodiments, when a force is applied, the length of the spring 502 extends and the distance between the coils increases. In still such embodiments, when the force is removed, the spring 502 may return to the resting position with the coils close together. As will be appreciated, the spring 502 may be made of any suitable material.

In other embodiments, the body 412 may have discontinuous walls, as shown in FIG. 5B and FIG. 5C. The discontinuities 504 may span at least a portion of the length of the body 412. In some embodiments, the discontinuities 504 may run along the entire length of the body 412. In other embodiments, discontinuities 504 may be around the entire circumference of the body 412. In other embodiments, discontinuities 504 may only be around a portion of the circumference of the body 412. Although longitudinal discontinuities and circular discontinuities are shown in FIG. 5B and FIG. 5C, respectively, the discontinuities 504 may be of any suitable geometry. For example, the discontinuities 504 may be circular (as shown in FIG. 5C). Although one geometry of discontinuity is shown in each of the exemplary embodiments in FIG. 5B and FIG. 5C, any number of suitable geometries may be used. For example, in one embodiment, a portion of the body 412 may have circular discontinuities (as shown in FIG. 5C) and a portion of the body 412 may have longitudinal discontinuities (as shown in FIG. 5B).

In some embodiments, a plurality of tubular bodies 506 may connect the first end 414 and the second end 416, as shown in FIG. 5E. Although five tubular bodies 506 are shown in FIG. 5E, any suitable number of tubular bodies 506 may connect the first end 414 and the second end 416. In some embodiments, the tubular bodies 506 may be spaced apart equally around the circumference of the first end 414 and/or the second end 416. In other embodiments, tubular bodies 506 may be spaced apart un-equally. As will be appreciated, the tubular bodies 506 may be made of any suitable material. For example, tubular bodies 506 may be made of a material that extends temporarily when an axial force is applied. In some embodiments, the tubular bodies 506 may be made of the same material. In other embodiments, each of the plurality of tubular bodies 506 may be made of a different material. In some embodiments, the tubular bodies 506 may be springs.

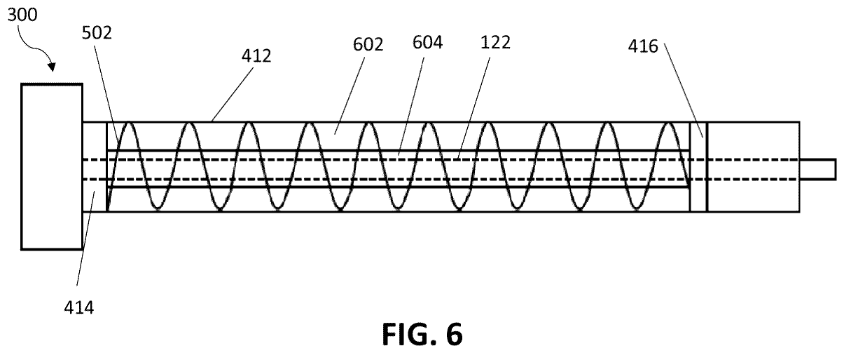

As shown in FIG. 6, the body 412 may comprise a first layer 602 and a second layer 604. The second layer 604 may be positioned between the first layer 602 and the catheter 122. In some embodiments, the second layer 604 may extend along the entire axial length between the first end 414 and the second end 416. In other embodiments, the first layer 602 and the second layer 604 may be different lengths. For example, the second layer 604 may extend along at least a portion of the axial length between the first end 414 and the second end 416. In some embodiments, the first layer 602 and the second layer 604 may be connected to both the first end 414 and the second end 416. In other embodiments, only one of the first layer 602 and the second layer 604 may be connected to the first end 414 and the second end 416. For example, in such other embodiments, the first layer 602 may be connected to the first end 414 and the second end 416, while the second layer 604 may be connected to only the first end 414.

Furthermore, in some embodiments, there may be a spring 502 positioned between the first layer 602 and the second layer 604. In other embodiments, the first layer 602 and the spring 502 may be made of a material that extends at a load less than the load required to relocate the portion of the body 412 in the repositioning unit 300. In some embodiments, the first layer 602, the second layer 604 and the spring 502 may be made of a material that extends at a load less than the load required to relocate the portion of the body 412 in the repositioning unit 300. In other embodiments, the first layer 602 and the second layer 604 may be made of the same material. In some embodiments, the first layer 602 and the second layer 604 may be made of different materials. For example, the first layer 602 may be made of a material that deforms at a load less than the load required to deform the material of the second layer 604.

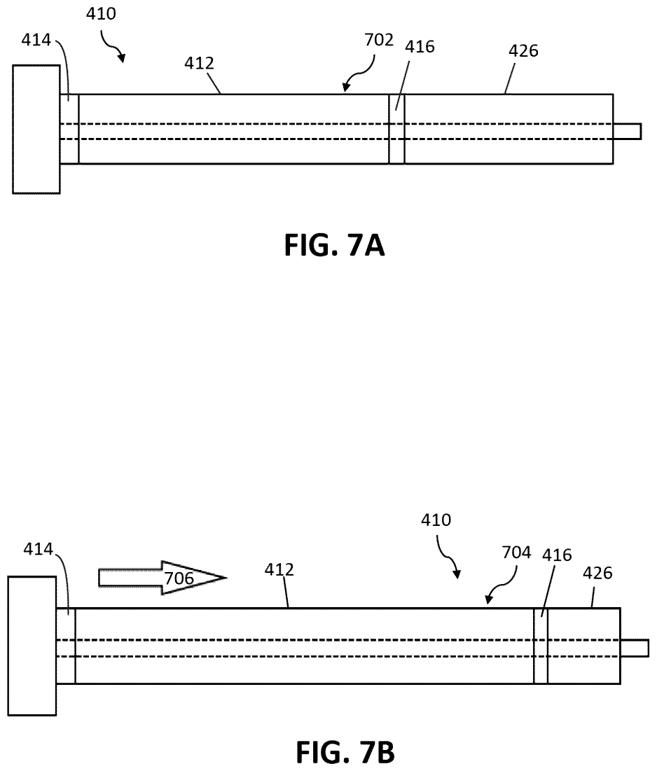

Overall, the accessory 410 may have two positions—a first position 702 and a second position 704 as shown in FIG. 7A and FIG. 7B, respectively. When there is no force 706 applied and the accessory 410 is at rest, the accessory 410 may be in a first position 702 as shown in FIG. 7A. When there is a force 706 applied to the accessory 410, the accessory 410 may be in a second position 704 as shown in FIG. 7B. When the accessory 410 is in the second position 704, the length of the body 412 extends as a result of the force 706. When the force 706 is removed, the accessory 410 may return to the first position 702. The accessory 410 returns to the first position 702 because the body 412 contracts once the force 706 is removed. In some embodiments, the body 412 may remain extended even after the force 706 is removed. In some embodiments, the first end 414 and the second end 416 may be attached in the first position 702. In still such embodiments, when the force 706 is applied, the first end 414 and the second end 416 may become disconnected as a result of the force 706 and the body 412 will extend. In some embodiments, the length of the anti-contamination sleeve 426 may not extend in the second position 704, but rather may be axially displaced along the axis of the catheter. In other embodiments, the length of the anti-contamination sleeve 426 may extend in the second position 704.

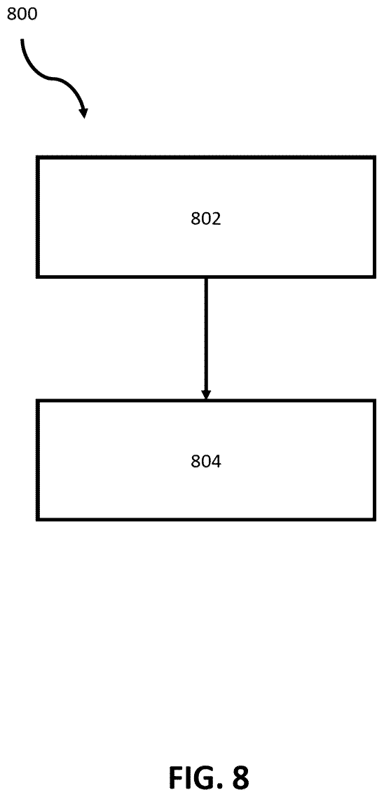

FIG. 8 illustrates a method 800 of preventing catheter dislocation according to some aspects of the present disclosure. In some embodiments, the method 800 may include the first step of applying 802 a force to an accessory to a blood pump system. The accessory may have a body configured to be slidable over a catheter. The body may have a first end configured to be attached to a portion of the blood pump system and a second end. The second end may be moveable relative to the first end. According to some embodiments, the first end may be attached to a repositioning unit. The method 800 may also include the step of moving 804 the accessory in a first direction while the blood pump system does not move in the first direction.

According to some embodiments, the method 800 may further include the step of detaching the first end and the second end. In still such embodiments, the first end may remain attached to the portion of the blood pump system and the second end is movable relative to the first end.

According to some embodiments, the method 800 may further include the step of attaching the accessory to the repositioning unit before moving 804 the accessory in a first direction.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Various embodiments are described below.

In a first embodiment, an accessory for use with a blood pump system may be provided. The blood pump system may include a blood pump, a catheter attached to the blood pump, and a repositioning unit configured to receive at least a portion of the blood pump and/or catheter and to secure the blood pump system to the patient. The accessory includes (i) a first end configured to be attached to a portion of a repositioning unit; and (ii) a second end, wherein when the accessory is grasped and when a force is applied to the accessory, the accessory is moveable in a first direction of the force while the blood pump system does not move in the first direction.

In a second embodiment, based on the first embodiment, the accessory further includes a first position and a second position, wherein the accessory is in the first position when no force is applied, and the accessory is in the second position when force is applied.

In a third embodiment, based on the second embodiment, the accessory returns to the first position when force is removed.

In a fourth embodiment, based on any of the first through third embodiments, the first end and second end are removably attached, and the second end is detached from the first end when the accessory is grasped and when a force is applied to the accessory.

In a fifth embodiment, based on any of the first through fourth embodiments, the accessory further includes a body, wherein the body extends along the longitudinal axis of the catheter and connects the first end and the second end.

In a sixth embodiment, based on the fifth embodiment, the body comprises a distal end and a proximal end connected by a tubular structure.

In a seventh embodiment, based on the sixth embodiment, the walls of the body are discontinuous along at least a portion of the longitudinal length of the catheter.

In an eighth embodiment, based on any of the fifth through seventh embodiments, the body comprises at least one tubular body connecting the first end and the second end.

In a ninth embodiment, based on the eighth embodiment, the at least one tubular body comprises a plurality of tubular bodies connecting the first end and the second end.

In a tenth embodiment, based on any of the first through ninth embodiments, the repositioning unit comprises a sheath configured to be inserted into a blood vessel of a patient through a vessel aperture and a tubular body configured for insertion of a catheter therethrough.

In an eleventh embodiment, based on the tenth embodiment, at least a portion of the sheath is positioned in a blood vessel of the patient.

In a twelfth embodiment, an accessory for use with a blood pump system having a blood pump, a catheter attached to the blood pump, a repositioning unit configured to receive at least a portion of the blood pump and/or catheter and to secure the catheter to the patient may be provided, the accessory including a body configured to be slidable over the catheter, wherein the body has a first end configured to be attached to a portion of the blood pump system and a second end, wherein the second end of the body is moveable relative to the first end upon application of a force.

In a thirteenth embodiment, based on the twelfth embodiment, the first end is attached to a repositioning unit.

In a fourteenth embodiment, based on the twelfth or thirteenth embodiment, the first end is integrally formed with the repositioning unit.

In a fifteenth embodiment, based on any of the twelfth through fourteenth embodiments, the repositioning unit comprises a sheath having a tubular body, the tubular body defining a lumen for insertion of a catheter therethrough.

In a sixteenth embodiment, based on any of the twelfth through fifteenth embodiments, the repositioning unit comprises a proximal body and a distal body, the proximal body and distal body together defining a lumen for insertion of a catheter therethrough.

In a seventeenth embodiment, based on the sixteenth embodiment, at least a portion of the distal body is configured to be inserted into a patient's body to allow for a medical device to be inserted through the lumen into the patient's body.

In an eighteenth embodiment, based on any of the twelfth through seventeenth embodiments, a distal end and a proximal end are positioned on opposite ends of the body.

In a nineteenth embodiment, based on the eighteenth embodiment, the body has a first layer and a second layer, the second layer positioned between the first layer and the catheter.

In a twentieth embodiment, based on the nineteenth embodiment, the accessory further includes a spring positioned between the first layer and the second layer.

In a twenty-first embodiment, based on the twentieth embodiment, the first layer and the spring are made of a material that extends at a load less than the load required to relocate the portion of the body in the repositioning unit.

In a twenty-second embodiment, based on any of the eighteenth through twenty-first embodiments, the body is configured to be a spring.

In a twenty-third embodiment, based on the twenty-second embodiment, the spring is made of a metal material.

In a twenty-fourth embodiment, based on the twenty-second embodiment, the spring is made of a polymer material.

In a twenty-fifth embodiment, based on any one of eighteenth through twenty-fourth embodiments, the proximal and distal end are removably connected.

In a twenty-sixth embodiment, based on the twenty-fifth embodiment, the body is configured to extend when the proximal end is disconnected from the distal end.

In a twenty-seventh embodiment, based on any of the eighteenth through twenty-sixth embodiments, the distal end is removably connected to the repositioning unit.

In a twenty-eighth embodiment, based on any of the twelfth through twenty-seventh embodiments, the body further includes a proximal portion, a distal portion, and an intermediate portion extending between the proximal portion and the distal portion.

In a twenty-ninth embodiment, based on the twenty-eighth embodiment, the proximal portion and the distal portion are made of a viscoelastic material.

In a thirtieth embodiment, based on the twenty-ninth embodiment, the intermediate portion is made of an elastic polymer material.

In a thirty-first embodiment, based on any one of the twenty-eighth through thirtieth embodiments, the proximal portion and the distal portion are made of the same material.

In a thirty-second embodiment, based on any one of the twenty-eighth through thirtieth embodiments, the proximal portion is made of a first material, the distal portion is made of a second material, and the intermediate portion is made of a third material. At least one of the first, second, or third materials may be different from another of the first, second, or third materials. Preferably, all three materials are different.

In a thirty-third embodiment, based on the thirty-second embodiment, the first material extends at a load less than the load required to extend the third material.

In a thirty-fourth embodiment, based on the thirty-second or thirty-third embodiment, the third material extends at a load less than the load required to extend the second material.

In a thirty-fifth embodiment, based on any one of the twenty-eighth through the thirty-fourth embodiment, the proximal portion and the distal portion are made of an elastic polymer material.

In a thirty-sixth embodiment, based on the thirty-fifth embodiment, the intermediate portion is made of a viscoelastic material.

In a thirty-seventh embodiment, based on any of the twelfth through the thirty-sixth embodiments, the body is made of a material that extends at a load less than the load required to relocate the portion of the body in the repositioning unit.

In a thirty-eighth embodiment, a method may be provided. The method may include: (i) applying a force to an accessory to a blood pump system, the accessory having a body configured to be slidable over a catheter, wherein the body has a first end configured to be attached to a portion of the blood pump system and a second end, wherein the second end of the body is moveable relative to the first end; and (ii) moving the accessory in a first direction while the blood pump system does not move in the first direction.

In a thirty-ninth embodiment, based on the thirty-eighth embodiment, the method further includes the step of detaching the first end and the second end, wherein the first end remains attached to the portion of the blood pump system and the second end is moveable relative to the first end.

In a fourtieth embodiment, based on the thirty-eighth or thirty-ninth embodiment, the first end is attached to a repositioning unit.

In a forty-first embodiment, based on any of the thirty-eighth through fourtieth embodiments, the method further includes the step of attaching the accessory to the repositioning unit.

In a forty-second embodiment, a system for use with a catheter-based blood pump may be provided. The system may include: (i) a blood pump, the blood pump having an inlet, an outlet, an impeller, and a cannula; (ii) a catheter having a proximal end and a distal end, the distal end connected to the blood pump; (iii) a repositioning unit, at least a portion of the catheter positioned across the repositioning unit; and (iv) an accessory, the accessory comprising a body configured to be slidable over the catheter, wherein the body has a first end configured to be attached to the repositioning unit and a second end, wherein the second end is moveable relative to the first end upon application of a force.

In a forty-third embodiment, based on the forty-second embodiment, the repositioning unit comprises a sheath having a tubular body, the tubular body defining a lumen configured to allow the catheter to pass through.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. An accessory for use with a blood pump system, the blood pump system having a blood pump, a catheter attached to the blood pump, and a repositioning unit configured to receive at least a portion of the blood pump and/or catheter and to secure the blood pump system to the patient, the accessory comprising:
a first end configured to be attached to a portion of a repositioning unit; and
a second end, wherein when the accessory is grasped and when a force is applied to the accessory, the accessory is moveable in a first direction of the force while the blood pump system does not move in the first direction.

2. The accessory of claim 1, further comprising a first position and a second position, wherein the accessory is in the first position when no force is applied, and the accessory is in the second position when force is applied.

3. The accessory of claim 2, wherein the accessory returns to the first position when force is removed.

4. The accessory of claim 1, wherein the first end and second end are removably attached, and the second end is detached from the first end when the accessory is grasped and when a force is applied to the accessory.

5. The accessory of claim 1, further comprising a body, wherein the body extends along the longitudinal axis of the catheter and connects the first end and the second end.

6. The accessory of claim 5, wherein the body comprises a distal end and a proximal end connected by a tubular structure.

7. The accessory of claim 6, wherein the walls of the body are discontinuous along at least a portion of the longitudinal length of the catheter.

8. The accessory of claim 5, wherein the body comprises at least one tubular body connecting the first end and the second end.

9. The accessory of claim 5, wherein the tubular body comprises a plurality of tubular bodies connecting the first end and the second end.

10. The accessory of claim 1, wherein the repositioning unit comprises a sheath configured to be inserted into a blood vessel of a patient through a vessel aperture and a tubular body configured for insertion of a catheter therethrough.

11. The accessory of claim 10, wherein at least a portion of the sheath is positioned in a blood vessel of the patient.

12. An accessory for use with a blood pump system having a blood pump, a catheter attached to the blood pump, a repositioning unit configured to receive at least a portion of the blood pump and/or catheter and to secure the catheter to the patient, the accessory comprising:
a body configured to be slidable over the catheter, wherein the body has a first end configured to be attached to a portion of the blood pump system and a second end, wherein the second end of the body is moveable relative to the first end upon application of a force.

13. The accessory of claim 12, wherein the first end is attached to a repositioning unit.

14. The accessory of claim 12, wherein a proximal end and a distal end are removably connected.

15. The accessory of claim 14, wherein the body is configured to extend when the proximal end is disconnected from the distal end.

16. The accessory of claim 12, wherein the proximal portion is made of a first material, the distal portion is made of a second material, and the intermediate portion is made of a third material.

17. The accessory of claim 16, wherein the first material extends at a load less than the load required to extend the third material.

18. The accessory of claim 12, wherein the body is made of a material that extends at a load less than the load required to relocate the portion of the body in the repositioning unit.

19. A method comprising:
applying a force to an accessory to a blood pump system, the accessory having a body configured to be slidable over a catheter, wherein the body has a first end configured to be attached to a portion of the blood pump system and a second end, wherein the second end of the body is moveable relative to the first end; and
moving the accessory in a first direction while the blood pump system does not move in the first direction.

20. A system for use with a catheter-based blood pump, comprising:
a blood pump, the blood pump having an inlet, an outlet, an impeller, and a cannula;
a catheter having a proximal end and a distal end, the distal end connected to the blood pump;
a repositioning unit, at least a portion of the catheter positioned across the repositioning unit; and
an accessory, the accessory comprising a body configured to be slidable over the catheter, wherein the body has a first end configured to be attached to the repositioning unit and a second end, wherein the second end is moveable relative to the first end upon application of a force.
